(19) United States
(12) Patent Application Publication
(10) Pub. No.: US 2026/0057987 A1
(43) Pub. Date: Feb. 26, 2026

(54) SYSTEM AND METHOD FOR AUTOMATING IDENTIFICATION OF TARGETED PATIENTS AND MEDICATION SYNCHRONIZATION

(71) Applicant: Omnicell, Inc., Fort Worth, TX (US)

(72) Inventors: Ryan Moore, Mars, PA (US); Maria Garces, Tampa, FL (US); James F. Boericke, JR., Raleigh, NC (US)

(73) Assignee: Omnicell, Inc., Fort Worth, TX (US)

(21) Appl. No.: 19/301,787

(22) Filed: Aug. 15, 2025

Related U.S. Application Data
(60) Provisional application No. 63/684,100, filed on Aug. 16, 2024.

(51) Int. Cl. G16H 20/10

(52) U.S. Cl. CPC G16H 20/10

(57) ABSTRACT

A system for automating prescription medication synchronization, including: a processor, a memory storing instructions that, when executed by the processor, cause the system to: retrieve prescription data for a patient; verify a use agreement; authenticate the patient; determine eligibility for a synchronization program; receive a patient opt-in; and generate a medication synchronization schedule.

### CROSS-REFERENCE TO RELATED APPLICATIONS

This application claims the benefit of U.S. Provisional Patent Application No. 63/684,100, filed Aug. 16, 2024, the entire disclosure of which is hereby incorporated by reference herein.

### TECHNICAL FIELD

Aspects of the present disclosure generally relate to prescription medications, and more particularly, to systems and methods for automating identification of targeted patients and synchronization of medications.

### BACKGROUND

Reference may be made herein to other United States patents, foreign patents, and/or other technical references. Any reference made herein to other documents is an express incorporation by reference of the document so referenced in its entirety.

Management of multiple medication prescriptions may be a significant challenge for many patients, particularly those with chronic conditions requiring continuous, reoccurring, and complex medication regimens. Patients often face difficulties in synchronizing or timely fulfilling their prescriptions, which can lead to missed doses, incorrect medication timing, and potential adverse drug interactions. These issues are exacerbated when prescriptions have varying refill dates, requiring patients to make multiple trips to the pharmacy and increasing the likelihood of non-adherence to their individual, prescribed medication regimens.

Conventional prescription practices typically involve patients receiving individual prescriptions from their healthcare providers, which are then filled at a pharmacy. Each prescription is generally managed independently, with its own specific refill schedule. As a result, patients may need to visit the pharmacy multiple times per month to refill different medications. This process is not only inconvenient but also increases the risk of errors and non-compliance, particularly for elderly patients, those with mobility issues, or even overly busy individuals. Thus, prescription synchronization is a critical component for ensuring patients correctly follow their prescribed medication regimen.

Current systems and methods for prescription synchronization often require patients to meet with a pharmacist in person to discuss their medication schedules and adjust refill dates accordingly. While this can be an effective method for aligning prescription refill schedules, it is time-consuming and may not be practical for all patients, especially those living in rural or underserved areas. Additionally, the reliance on in-person meetings does not accommodate patients' needs for flexibility and convenience in managing their prescriptions.

This problem extends beyond patient inconvenience; it also places a significant burden on the pharmacy and pharmacy staff. Pharmacists and pharmacy technicians must allocate considerable time and resources to manage and coordinate individual prescription schedules manually. This process includes reviewing patient records, contacting healthcare providers, and conducting consultations to align refill dates. As a result, pharmacy staff are diverted from other critical tasks, such as providing patient counseling, managing inventory, and ensuring the accuracy and safety of dispensed medications. The inefficiencies inherent in current systems can lead to increased operational costs and reduce the overall capacity of pharmacies to effectively serve their communities.

There is a clear need for an improved system and method that allows patients to synchronize their prescriptions seamlessly, without the necessity of frequent visits to the pharmacy. Such a system and method would enhance medication adherence, reduce the burden on patients and pharmacists, and improve overall health outcomes by ensuring that patients take their medications as prescribed and on time.

To surmount these challenges, there is a need for a solution that not only automates the medication prescription assignment process but also leverages data analysis to optimize medication storage and access arrangements. By integrating data-driven methodologies and automation, the proposed system and method can mitigate delays, eliminate redundancies and mistakes, improve medication adherence, and enhance the overall efficiency of medication maintenance and distribution.

### SUMMARY

Techniques described herein are directed to prescription medications, and more particularly, to systems and methods for automating identification of targeted patients and synchronization of medications.

An example embodiment of the present disclosure may be a method for synchronizing prescription medications for a patient, comprising: receiving, by a computing system, prescription data associated with a patient; verifying a use agreement; authenticating the patient; determining eligibility for a prescription synchronization program; receiving an opt-in from the patient; and synchronizing a plurality of medications according to a computed refill alignment schedule.

In one embodiment, the method further comprises determining the refill alignment schedule based on at least one of: current refill dates, medication dosages, and refill intervals.

In one embodiment, synchronizing the plurality of medications comprises generating partial fill information for one or more medications to align with other medication refill dates.

In one embodiment, the method further comprises providing the patient with a confirmation card comprising a pick-up date, pharmacy address, and synchronized medication list.

In one embodiment, the method further comprises authenticating the patient comprises validating inputted identification information against pharmacy records.

In one embodiment, determining eligibility comprises applying patient-level and medication-level exclusion criteria.

In one embodiment, the patient-level exclusion criteria comprise excluding patients who are inactive, deceased, or hospitalized.

In one embodiment, the medication-level exclusion criteria comprise excluding medications with insufficient day supply or lapsed prescriptions.

In one embodiment, the method further comprises filtering patients who are enrolled, partially enrolled, or have opted out of the synchronization program.

In one embodiment, the use agreement and patient interaction are presented via a chatbot interface communicatively coupled to a patient device.

An example embodiment of the present disclosure may be a system for automating prescription medication synchronization, comprising: a processor; a memory storing instructions that, when executed by the processor, cause the system to: retrieve prescription data for a patient; verify a use agreement; authenticate the patient; determine eligibility for a synchronization program; receive a patient opt-in; and generate a medication synchronization schedule.

In one embodiment, the system further comprises a user interface configured to display opt-in options and synchronization details to the patient.

In one embodiment, the synchronization schedule aligns medication refill dates by calculating partial fills.

In one embodiment, the system is configured to exclude patients or medications based on eligibility criteria stored in a pharmacy database.

In one embodiment, the eligibility criteria comprise patient age, enrollment status, activity status, and medication history.

In one embodiment, the system further comprises a chatbot module configured to manage patient interactions and secure identification processes.

In one embodiment, the system further comprises a medication alignment module configured to compute optimal synchronization dates across multiple prescriptions.

In one embodiment, the system further comprises a database configured to store confirmation messages including synchronized medication lists and pharmacy pickup instructions.

In one embodiment, the processor is further configured to generate instructions for a pharmacy management system to dispense medications according to the synchronization schedule.

In one embodiment, the system communicates with a cloud-based infrastructure to exchange data with at least one of: a pharmacy system, a patient device, and a healthcare provider system.

The above summary has outlined, rather broadly, some features and technical advantages of the present disclosure so that the detailed description that follows may be better understood. Additional features and advantages of this disclosure will be described below. It should be appreciated by those skilled in the art that this disclosure may be readily utilized as a basis for modifying or designing other systems and structures for carrying out the same or similar purposes of the present disclosure. It should also be realized by those skilled in the art that such equivalent systems and structures do not depart from the teachings of the disclosure as set forth in the appended claims. The novel features, which are believed to be characteristic of this disclosure, both as to its organization and method of operation, together with further features and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The features, nature, and advantages of the present disclosure will become more apparent from the detailed description set forth below when taken in conjunction with the drawings, in which:

FIG. 1 depicts an illustrative automated medication synchronization system according to an embodiment of the present disclosure;

FIG. 2 depicts an illustrative method for enrolling a patient in an automated medication synchronization system according to an embodiment of the present disclosure;

FIG. 3 depicts an illustrative method for adding a medication without alignment according to an embodiment of the present disclosure;

FIG. 4 depicts an illustrative method for adding a medication with alignment according to an embodiment of the present disclosure;

FIG. 5 depicts an illustrative diagram showing data flow within an automated enrollment and medication synchronization system according to an embodiment of the present disclosure;

FIG. 6 depicts an illustrative method for generating a list of patients enrolled in the medication synchronization program that are likely to add a medication to their existing synchronized medications program, according to an embodiment of the present disclosure;

FIG. 7 depicts an illustrative method for determining eligible patients for enrolling into the medication synchronization program, according to an embodiment of the present disclosure; and

FIG. 8 depicts an illustrative system for automated patient enrollment and medication synchronization system according to an embodiment of the present disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent, however, to those skilled in the art that these concepts may be practiced without these specific details. In some instances, well-known features may be omitted or simplified in order not to obscure the embodiment being described.

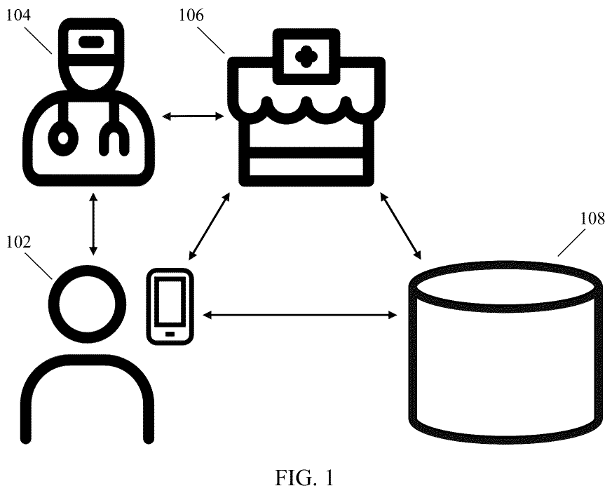

FIG. 1 depicts an illustrative automated medication synchronization system according to an embodiment of the present disclosure. An automated prescription medication synchronization system and its interaction with various entities involved in the prescription process is shown, including a user or patient 102, a doctor 104, a pharmacy 106, and automated authentication and medication synchronization system 108.

User 102 may be a patient who requires medication and interacts with the healthcare system to obtain and manage prescriptions. This user may, for example, have multiple chronic conditions necessitating the use of several medications with different refill schedules, making synchronization critical to maintaining adherence to a prescription medication regimen. Doctor 104 may be a healthcare provider who prescribes medications to user 102, evaluates user's 102 health conditions and determines the appropriate medications needed for treatment. Doctor 104 may issue prescriptions which are then communicated physically or electronically to pharmacy 106. Pharmacy 106 may be a facility where the prescribed medications are filled and dispensed and is responsible for receiving prescription information from doctor 104, storing this information in a pharmacy database, and dispensing the medications to user 102. This database may be part of a larger pharmacy management system that tracks inventory, patient records, prescription histories, etc. Automated authentication and medication synchronization system 108 may be responsible for retrieving and verifying prescription information, ensuring user eligibility, and synchronizing user prescriptions to streamline the refill process. This system integrates with the pharmacy's database, and utilizes advanced algorithms to align refill dates and manage communication with users 102.

The interaction begins with doctor 104 prescribing a medication to user 102 and communicating the prescription information to pharmacy 106. This may include sending an electronic prescription from the doctor's office to the pharmacy's system, or any other means of communicating a prescription known in the art. Pharmacy 106 receives the prescription information from doctor 104 and stores the prescription information in a database, which tracks user prescriptions, refill dates, medication inventory, etc. Automated authentication and medication synchronization system 108 accesses the database of pharmacy 106 to retrieve user information and associated prescription details. This involves secure data retrieval processes to ensure patient privacy and data integrity. System 108 verifies user's 102 eligibility for the synchronization program, ensuring that user 102 meets the criteria for participation. Eligibility requirements will be discussed in further detail below.

System 108 may contact user 102 to inform them about the synchronization program and its benefits. This contact can be made via various communication channels, including phone calls, text messages, emails, or through a dedicated mobile application, providing flexibility and convenience for user 102. In a preferred embodiment, system 108 communicates to user 102 via text message. User 102 confirms their identity using secure authentication methods and opts into using the synchronization program. This step ensures the identity of user 102 is confirmed, and user 102 is aware of and consents to utilizing system 108.

System 108 utilizes user prescription information to synchronize the refill dates of all medications. This involves adjusting the refill schedules to allow user 102 to pick up all prescriptions at the same time, reducing the number of trips to pharmacy 106. The system takes into account factors such as medication dosages, refill intervals, and insurance constraints to create an optimal synchronization plan. Existing prescriptions may also be partially filled according to the needs of user 102. The synchronized prescription information is sent to pharmacy 106 by system 108. This information includes the updated refill schedule and any special instructions for the pharmacy staff. Pharmacy 106 fills the synchronized prescriptions according to the new schedule, preparing all medications for a single pickup.

Finally, user 102 picks up all synchronized prescriptions from pharmacy 106 at the designated date and time. This process reduces the frequency of pharmacy visits, minimizes the risk of missed doses, and enhances overall medication adherence. The medication synchronization also allows pharmacy staff to manage the workflow more efficiently, as multiple prescriptions may be prepared at once, reducing operational strain.

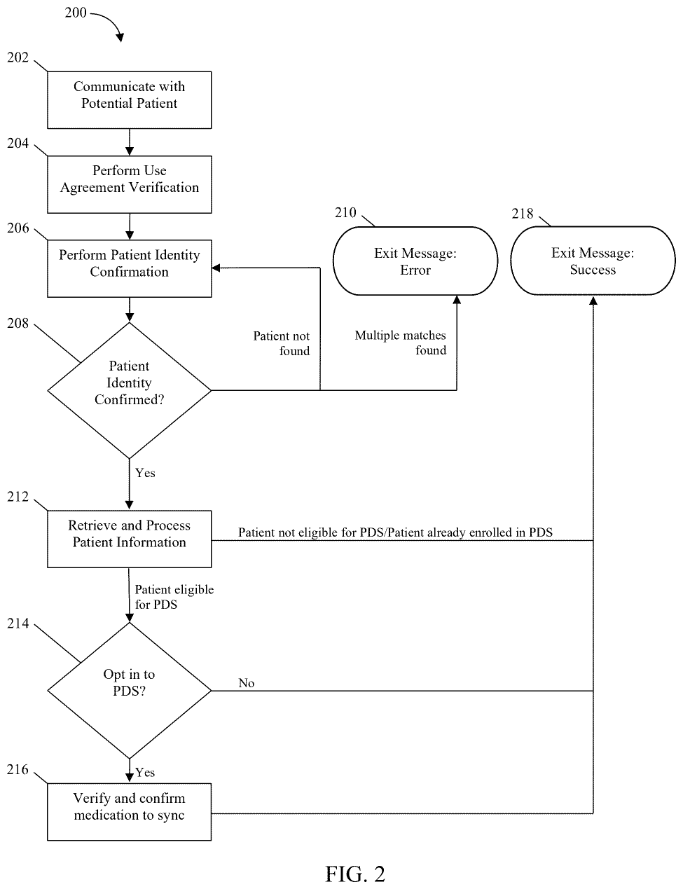

FIG. 2 depicts an illustrative method for enrolling a patient in an automated medication synchronization system according to an embodiment of the present disclosure. Method 200 outlines a comprehensive process for enrolling a patient or user in a medication synchronization program. The steps of this method are detailed as follows.

At step 202, a user or patient 102 receives an invite to enroll in the medication synchronization program. The invitation may be sent via email, SMS, or through a mobile application notification, providing a direct link to begin the enrollment process. The patient may accept the invitation (e.g., using the direct link provided by clicking it) and begin the enrollment process. In one embodiment, a chatbot interface may be presented to the patient after selection of the direct link in order to facilitate the synchronization program. The patient may be presented with an introduction message about synchronizing their medications. The message may, for example, without limitation, explain the benefits of medication synchronization, inform the patient that the program is only valid for certain prescriptions, inform the patient of general prescription cost details, etc.

At step 204, use agreement verification may be performed. The patient may be presented with a use agreement outlining the terms and conditions of participating in the synchronization program, ensuring the patient understands their rights and responsibilities and terms and conditions for using the program. After the patient agrees to the use agreement, at step 206, the patient's identity may be confirmed. The patient may be prompted to confirm their identity, for example, by entering their date of birth (DOB), social security number, other identifying information, or other virtual identification means. This step ensures that the system correctly identifies the patient. At step 208, if patient identification is unsuccessful (e.g., the patient is not found), an error message may be displayed to the patient, and method 200 may proceed back to step 206. The system attempts to locate the patient in the database again, allowing for corrections in case of initial input errors. If multiple matches are found for the patient's identification information, the patient may be prompted for additional identification information, and if unsuccessful, method 200 may exit at step 210, where an error message may be displayed to the patient.

If the patient's identity is confirmed, method 200 may proceed to step 212 and the patient's information may be retrieved and processed. If the patient is found to not be eligible for patient data synchronization (PDS) or is already enrolled in PDS, method 200 may terminate at step 218 with an appropriate exit message. For example, without limitation, a patient who is already enrolled in PDS may be updated with medication status, pickup information, etc. If a patient is not eligible for PDS, the ineligibility may be explained to the patient, and the patient may be given contact information for further assistance.

At step 214, if patient is eligible for PDS, the patient is presented with an acknowledgment message that prompts the patient to opt in/out of PDS. This message confirms the patient's eligibility and asks if they wish to participate in the synchronization program. At step 218, if the patient does not opt in, the method exits and the patient is provided with their current medication information. At step 216, if the patient opts in to the program, the patient is shown a list of medications to be synchronized and asked to confirm the medications to be synchronized. The patient may review their current medications and confirm which medications should be included in the synchronization program. The patient may decline to synchronize one or more of their current medications, after which method 200 terminates at step 218 and the patient is presented with medication information, pickup information, etc. In one embodiment, the patient's medication may be filled, and the patient may be given multiple pickup times.

If the patient confirms, the patient is shown a confirmation card including patient name, synchronized medications, pick-up date, and pharmacy address. This final step provides the patient with a summary of the synchronized medications, the scheduled pick-up date, and the location of the pharmacy. The pharmacy may then fill the synchronized prescriptions, including any partial fills, for patient to pick up.

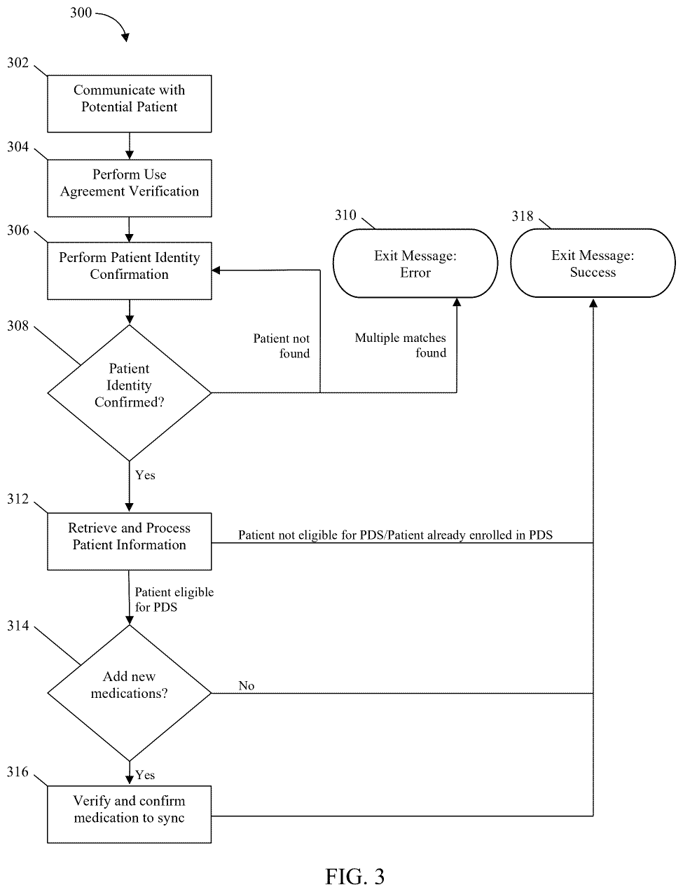

FIG. 3 depicts an illustrative method for adding a medication without alignment according to an embodiment of the present disclosure. Method 300 outlines a comprehensive process for a patient to add new medications to be synchronized using a system interface.

Method 300 begins with steps 302-312. Steps 302-312 are substantially similar to steps 202-212 of method 200, and relate to automated contacting of the patient, initiation of patient communication, use agreement acknowledgement, and patient verification.

At step 314, the patient is presented with an option to add new medications to be synchronized. This option informs the patient about the opportunity to include additional medications in the synchronization program. If the patient declines synchronization of the additional medications, method 300 may proceed to step 318, wherein the additional medications may be filled without synchronization, and medication information and pickup information may be displayed to the patient. At step 316, if the patient confirms addition of the new medication to PDS, medication information and potential pick up information may be communicated to the patient or confirmation. Once confirmed, at step 318, the medication may be filled with a synchronized pick-up date, and a confirmation card may be displayed with, for example, the patient name, partial fill information, pick-up date, and the store address. This card provides the patient with detailed information about the partial fill, including when and where the medications can be picked up. The patient may also be displayed a message that their medications will be synchronized after the partial fill, and all medications will be able to be picked up on the same date. The message ensures the patient understands that the synchronization process will be completed after the partial fill is picked up.

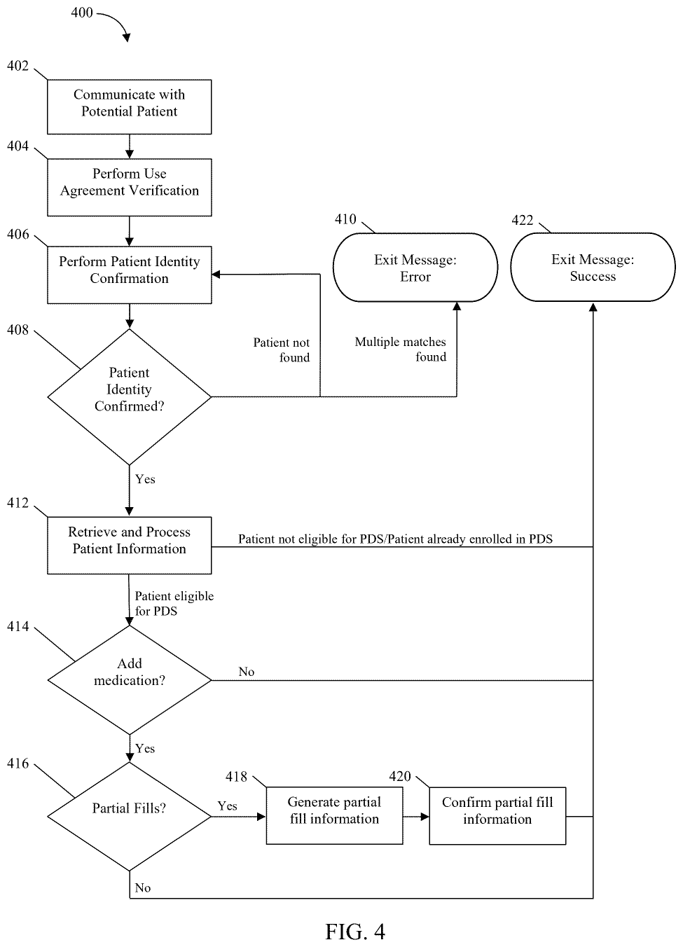

FIG. 4 depicts an illustrative method for adding a medication with alignment according to an embodiment of the present disclosure. Method 400 begins with steps 402-414. Steps 402-414 are substantially similar to steps 302-314 of method 300, and relate to automated contacting of the patient, initiation of the chatbot, use agreement acknowledgement, and patient verification.

At step 416, if the patient confirms addition of medication to PDS, the system checks if the medication requires partial fills. If the medication does not require a partial fill at step 416, the process exits with a custom exit message at step 422. In one embodiment, the custom exit message may be a confirmation card acknowledging addition of the new medication in the synchronization program and displaying pick-up information including the patient's name, medications to be picked up, pick-up date, and the pharmacy address. At step 418, if the new medication can be partially filled, partial fill information may be generated to maximize convenience for the user. For example, without limitation, if a medication to be partially filled is determined to be needed by the user as soon as possible, the prescription may be prioritized and an earlier partial fill pick-up date may be generated. The partial fill information may also be generated based on the patient's schedule, the pharmacy's existing pick-up schedule, etc.

At step 420, the patient receives the partial fill information. The partial fill information may include, for example, without limitation, the medications to be partially filled, the amount of each medication to be partially filled, the date for pick up of the partial fill, etc. The patient may then confirm the partial fill of their prescriptions. The system requests the patient's confirmation to proceed with the partial fill process after the partial fill information has been presented. The patient's medication may be partially filled to be aligned with their other medication enrolled in PDS. Once partial fill information is confirmed, method 400 may proceed to step 422 where medication information, pickup information, pharmacy information, etc. may be displayed to the patient, and the medication(s) may be filled and/or partially filled.

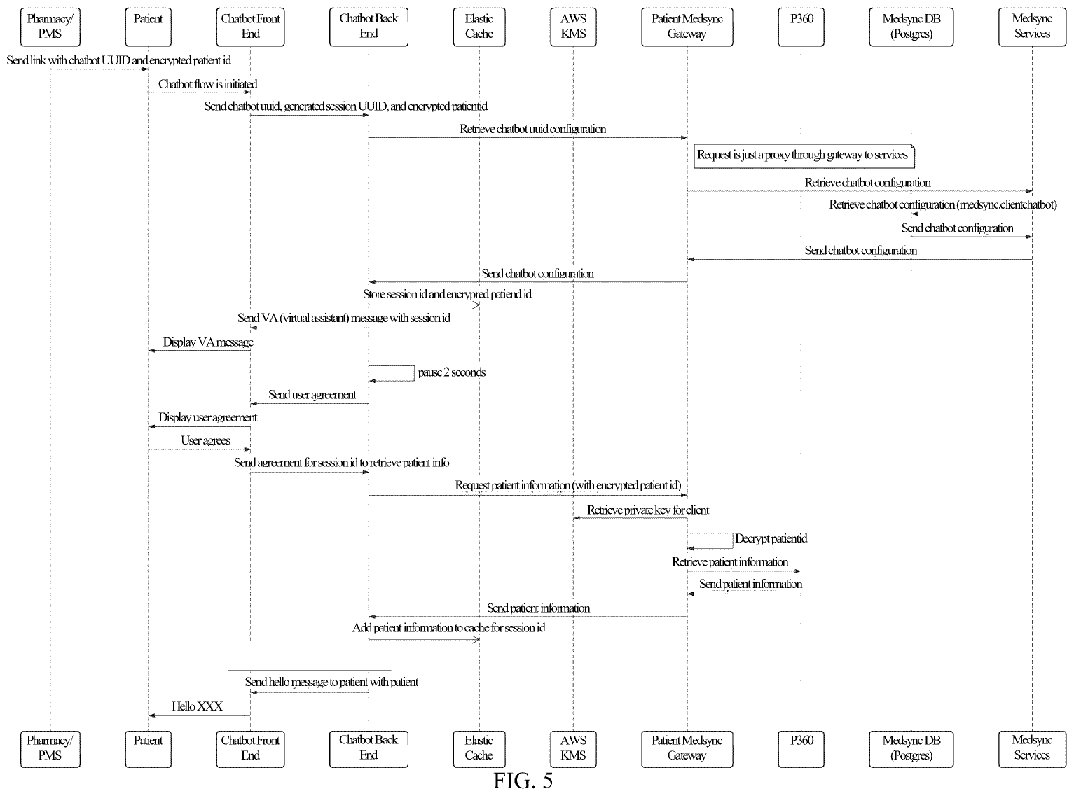

FIG. 5 depicts an illustrative diagram showing data flow within an automated enrollment and medication synchronization system according to an embodiment of the present disclosure. In particular, FIG. 5 shows data management within an automated identification and medication synchronization system for the initial contact and verification of a patient. As will be appreciated by one of skill in the art, the data flow shown is exemplary, and alternative data flow processes for contacting and verifying a patient fall within the scope of the present disclosure. The components of the system include Pharmacy Management System (PMS), patient, chatbot front end, chatbot back end, cache (e.g., ElasticCache), key management system (KMS), patient gateway, pharmacy database (e.g., P360), system database, and system services. The data flow proceeds as follows.

PMS sends a link with a chatbot UUID and an encrypted patient ID to the patient. This link is received by the patient who initiates the synchronization process. The patient clicks on the link, which triggers the initiation of the chatbot flow, activating chatbot front end. The chatbot front end sends the chatbot UUID, a generated session UUID, and the encrypted patient ID to chatbot back end. This step initiates the backend processes necessary to manage the patient's session and data. The chatbot back end sends a request to retrieve the chatbot UUID configuration from the patient gateway. The patient gateway sends a request to the system services to retrieve the chatbot configuration. The system services coordinate the retrieval of the required configuration data. The System services then sends a request to the system database to fetch the chatbot configuration. The system database holds the configuration details needed by the chatbot.

The system database then sends the chatbot configuration to system services. This configuration data is then relayed back through the system. The system services then sends the chatbot configuration to the patient gateway. The patient gateway then consolidates the configuration information and prepares the information for further transmission. The patient gateway then sends the chatbot configuration to the chatbot back end. The chatbot back end now has the necessary configuration to proceed with the interaction. The chatbot back end also stores the session ID and encrypted patient ID into cache. This ensures that session-specific information is readily accessible throughout the process.

The chatbot back end sends a virtual assistant message with the session ID to the chatbot front end. This message begins the interactive process with the patient. The message may include, for example, without limitation, information about the synchronization process, limitations of synchronization, etc. The chatbot front end then displays the virtual assistant message to the patient. The chatbot back end then sends a user agreement to the chatbot front end. This agreement outlines the terms and conditions for the synchronization program. The chatbot front end then displays the user agreement to the patient. The patient interacts with the chatbot front end to agree to the user agreement. The patient's agreement is captured and sent back to the backend system. The chatbot front end then sends the agreement for the session ID to the chatbot back end. This confirms the patient's consent for the synchronization process. The chatbot back end then requests patient information using the encrypted patient ID from patient gateway. This step is critical for retrieving and decrypting the patient's data.

The patient gateway then retrieves a private key for the patient from key management system. This key is necessary to decrypt the encrypted patient ID. The patient gateway then decrypts the patient ID using the retrieved private key. With the decrypted ID, the gateway can now request patient-specific information. The patient gateway then requests patient information from the pharmacy database. The pharmacy database contains the detailed records of the patient's prescriptions. The pharmacy database then sends patient information to the patient gateway. The information includes details necessary for the synchronization process, such as prescription information. The patient gateway then sends the patient information to the chatbot back end. The chatbot back end now has the comprehensive patient data required for the session.

The chatbot back end then stores the patient information for the session ID into cache. This ensures quick and efficient access to patient data during subsequent interactions. The chatbot back end then sends a custom intro message to the chatbot front end. This message provides a personalized greeting and instructions to the patient. The chatbot front end then displays the custom introduction message to the patient. The patient receives the message, completing the initial setup of the synchronization process and preparing for further interaction. This detailed description of data flow illustrates the complex yet streamlined process of managing patient interactions and medication synchronization through a sophisticated system architecture.

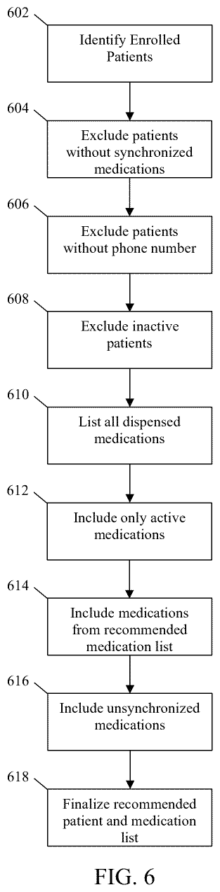

FIG. 6 depicts an illustrative method for generating a list of patients enrolled in the medication synchronization program that are likely to add a medication to their existing synchronized medications, according to an embodiment of the present disclosure. The steps of method 600 are as follows.

At step 602, all patients enrolled in the synchronization program are identified. This initial step involves retrieving a list of patients who are currently participating in the medication synchronization program from the relevant database.

At step 604, patients with no medication synchronized are excluded. From the list of identified patients, those who do not have any medications currently synchronized in the program are removed, ensuring that only patients with synchronized medications are considered.

At step 606, patients with no phone number are excluded. The list is further refined by removing patients who do not have a phone number on record, as a contact method is necessary for communication about the synchronization program.

At step 608, patients who are inactive, hospitalized, merged, or deceased are excluded. This step involves filtering out patients who are inactive in the system, currently hospitalized, whose records have been merged with others, or who have been marked as deceased, ensuring the focus is on eligible and reachable patients.

At step 610, all medications dispensed by the remaining patients are listed. For the remaining patients, a comprehensive list of all medications that have been dispensed to them is generated, capturing a complete medication profile.

At step 612, the active medication picked up in the last 120 days is selected. From the list of dispensed medications, only those that have been picked up by the patients within the last 120 days are included, ensuring the list focuses only on actively used medications.

At step 614, the medications from the recommended medication list are included. That is, medications that are part of a predefined recommended medication list, which may be more likely to be compatible with the medication synchronization program, are included.

At step 616, the medications which are not already synchronized in the synchronization medication program and have not been removed from the synchronization program in the last 120 days are included. Such medication may be eligible for the medication synchronization program.

At step 618, the final list of medications is generated. The filtered and refined list of medications that meet all the criteria set forth in the previous steps is produced, and the patients for which these medications are prescribed may be contacted to add the medication to the medication synchronization program.

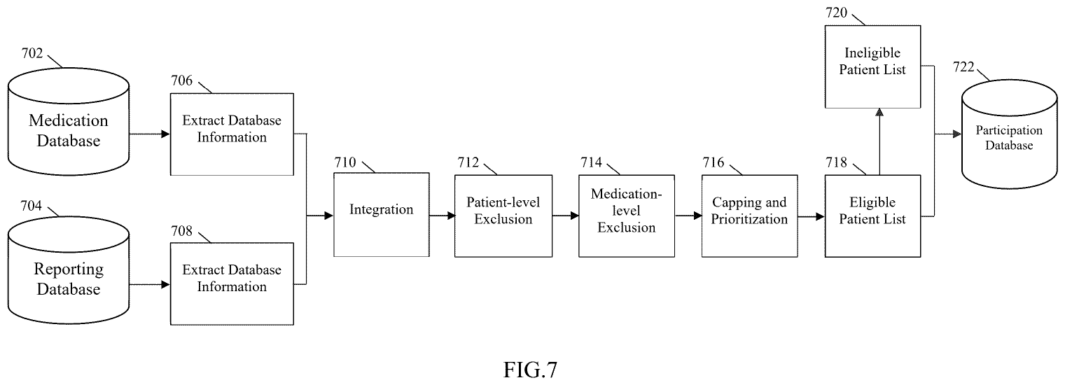

FIG. 7 depicts an illustrative method for determining eligible patients for enrolling into the medication synchronization program, according to an embodiment of the present disclosure. FIG. 7 illustrates a method for determining eligible patients for enrollment into a patient medication synchronization program by processing and integrating information from multiple databases and applying a series of exclusion criteria. The method comprises the following steps, each interacting with various system components to achieve the desired outcome.

Medication database 702 is the first database in the process. This database stores patient medication information in a normalized data format, which means that the data is organized to reduce redundancy and improve data integrity. At step 706, information is extracted from medication database 702. This step involves retrieving relevant patient medication data from the database, which will be used for further processing and evaluation.

Reporting database 704 is the second database utilized in the process. Unlike medication database 702, reporting database 704 stores patient data in a flattened format. In a flattened format, the data is denormalized, meaning it is organized to allow for faster querying and reporting, though it may contain more redundancy. At step 708, information is extracted from reporting database 704. This step retrieves patient data necessary for the synchronization process, which complements the data extracted from medication database 702.

The extracted data from both databases is then integrated at step 710. During this integration step, the system consolidates the normalized data from medication database 702 and the flattened data from reporting database 704 into a unified data model. Despite the differences in data format, the integration process aligns the data based on the shared patient model, ensuring consistency and completeness of the patient information.

Following data integration, patient-level exclusion occurs at step 712. This step involves filtering out patients who do not meet the eligibility criteria for enrollment into the medication synchronization program. The patients excluded at this step include those with a null patient ID, which indicates missing or incomplete patient information, and those outside of the specified age range, as age-specific programs might have different eligibility requirements. For example, patients under 18 years of age may be excluded. Long-term care patients and restricted plan patients are also excluded due to different medication management needs. Additionally, patients who are hospitalized, deceased, merged, or inactive are filtered out, as they are not suitable candidates for the synchronization program. An inactive patient, for example, may be a patient with no active medications within the last 120 days. Patients who have opted out or unenrolled more recently are excluded to respect their previous decisions, such as those who have opted out or unenrolled within the last year. Finally, patients who are already enrolled or partially enrolled in the synchronization program are excluded to avoid duplication.

After patient-level exclusions, medication-level exclusion takes place at step 714. In this step, the system filters out specific medications based on various criteria. Medications with a smaller day supply than required are excluded to ensure that only medications with sufficient coverage are considered. Medications with a lapsed prescription for the same drug are also excluded, as synchronization is intended for active and ongoing prescriptions. Additionally, medications outside a specified number of recommended medication ranges are excluded, which may involve ensuring that the patient is not prescribed an excessive or insufficient number of medications. The process may further restrict medications based on additional criteria such as day supply, the number of unbreakables (medications that cannot be split), and Medicaid months, which refer to the duration of Medicaid coverage.

Next, at step 716, the process proceeds to capping and prioritization. Capping is an optional feature that limits the maximum number of patients in a pharmacy to manage workload and resources effectively. When capping is enabled, prioritization is applied to ensure that more relevant patients have a higher priority for enrollment when the cap is reached. This prioritization allows the system to focus on patients who would benefit the most from synchronization, enhancing the program's effectiveness.

At step 718, the system generates two lists: an eligible patient list and an ineligible patient list. The eligible patient list includes all patients who passed the exclusion criteria and are ready for enrollment into the synchronization program. The ineligible patient list contains those who were excluded at any step of the process. Both lists are then uploaded to a participation database, where they can be accessed and used for further actions, such as enrolling eligible patients into the program or conducting follow-up with those who are ineligible.

FIG. 7 provides a comprehensive approach to determining patient eligibility for a synchronization program, integrating data from multiple sources, applying rigorous exclusion criteria, and ensuring that only the most suitable candidates are considered for enrollment. This method enhances the efficiency and effectiveness of the synchronization program by streamlining the patient selection process.

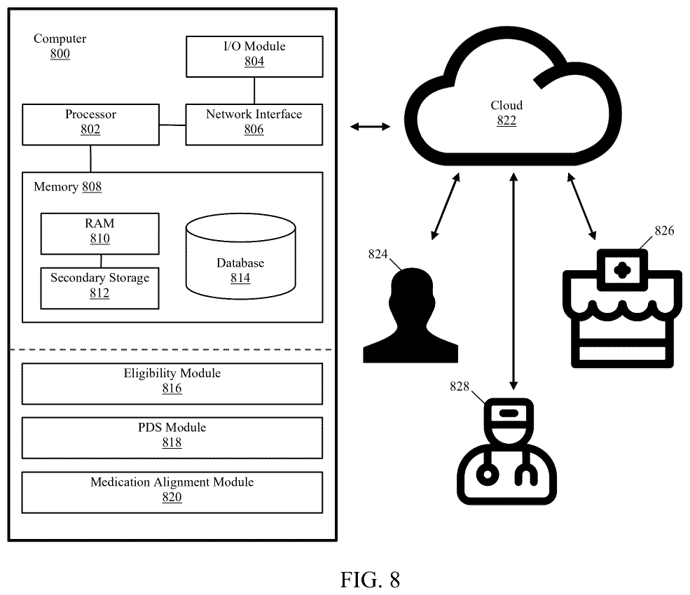

FIG. 8 depicts an illustrative system for automated patient enrollment and medication synchronization according to an embodiment of the present disclosure. This system is embodied in computer 800, which is a comprehensive computing device configured to perform complex operations necessary for determining patient and medication eligibility, synchronizing patient data, and aligning prescription medications.

Computer 800 comprises several interconnected components, each playing a crucial role in the overall system. Central to computer 800 is processor 802, which executes instructions and manages the operations of the system. Processor 802 interacts with and controls other components within computer 800, ensuring that data flows efficiently and processes are executed correctly. Input/output (I/O) module 804 facilitates communication between computer 800 and external devices, allowing the system to receive and send data necessary for patient data synchronization. I/O module 804 handles various types of input and output operations, such as receiving patient data, transmitting synchronization requests, and interacting with external entities like patients, doctors, and pharmacies. Network interface 806 provides connectivity between computer 800 and other systems or devices via cloud 822. This interface enables computer 800 to communicate with patient 824, doctor 828, and pharmacy 826, ensuring that data exchange occurs in a secure and efficient manner. The use of cloud 822 allows for scalable and flexible communication, supporting the high volume of data and interactions required by the PDS process.

Memory 808 is a critical component within computer 800, responsible for storing and managing data and instructions needed for operation. Memory 808 includes RAM 810, which provides temporary storage for data that processor 802 actively uses, allowing for quick access and processing of information. RAM 810 is essential for handling the real-time requirements of the PDS methodologies. Secondary storage 812 provides long-term data storage, holding large volumes of information that may not be in active use but are still essential for the system's operation. This includes data such as patient histories, medication records, and previous synchronization activities. Database 814 is a specialized component of secondary storage 812, structured to manage and organize data in a way that supports efficient retrieval and analysis. Database 814 stores patient and medication data in a format that allows for quick querying and manipulation, which is vital for the eligibility determination and synchronization processes.

Computer 800 may include various software modules that collectively facilitate the execution of complex tasks, data management, and process automation. These software modules are designed to be flexible, scalable, and adaptable to various applications, providing a robust foundation for a wide range of software-driven functionalities. Each software module may be a self-contained unit of software that encapsulates specific functionality or a set of related functions. These modules may interact with other modules and system components through different interfaces, allowing for modularity and ease of integration.

Eligibility module 816 is a specialized software component within computer 800 designed to determine patient and medication eligibility for participation in the PDS program. Eligibility module 816 implements the methodologies discussed in previous figures, such as patient-level and medication-level exclusions. By executing these methodologies, eligibility module 816 ensures that only appropriate candidates are selected for the synchronization program, improving the overall efficiency and effectiveness of the system.

PDS module 818 is responsible for performing the actual patient data synchronization process. PDS module 818 interacts with other components within computer 800 and with external entities via cloud 822 to align patient data, synchronize medication schedules, and ensure that all relevant information is up-to-date. PDS module 818 coordinates the retrieval, analysis, and synchronization of data, ensuring that patient medication schedules are aligned across multiple sources and that patients receive their medications at the correct times.

Medication alignment module 820 focuses on the specific task of aligning and filling prescription medications. This module ensures that medications are synchronized in a manner that aligns with patient needs and pharmacy capabilities. Medication alignment module 820 uses data from both eligibility module 816 and PDS module 818 to make informed decisions about how and when to fill prescriptions, optimizing the medication management process for both patients and pharmacies.

The overall architecture of computer 800, including its various modules and components, provides a technological framework for implementing the methodologies required for effective patient data synchronization.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the disclosure to the specific form or forms disclosed, but on the contrary, the intention is to cover all modifications, alternative constructions, and equivalents falling within the spirit and scope of the disclosure, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present

Although the present disclosure and its advantages have been described in detail, it should be understood that various changes, substitutions and alterations can be made herein without departing from the technology of the disclosure as defined by the appended claims. For example, relational terms, such as “above” and “below” and/or “inside” and “outside” are used with respect to a specific device. Of course, if the device is inverted, above becomes below, and vice versa. Additionally, if oriented sideways, above and below may refer to sides of a device. Further, reference to “first” or “second” instances of a feature, element, or device does not indicate that one device comes before or after the other listed device. Reference to first and/or second devices merely serves to distinguish one device that may be similar or similarly referenced with respect to another device.

Moreover, the scope of the present application is not intended to be limited to the particular configurations of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding configurations described herein may be utilized according to the present disclosure. Accordingly, the appended claims are intended to include within their scope such processes, machines, manufacture, compositions of matter, means, methods, or steps.

The description of the disclosure is provided to enable any person skilled in the art to make or use the disclosure. Various modifications to the disclosure will be readily apparent to those reasonably skilled in the art, and the generic principles defined herein may be applied to other variations without departing from the spirit or scope of the disclosure. Thus, the present disclosure is not intended to be limited to the examples and designs described herein but is to be accorded the widest scope consistent with the principles and novel features disclosed herein. Accordingly, the disclosure is not to be limited by the examples presented herein, but is envisioned as encompassing the scope described in the appended claims and the full range of equivalents of the appended claims.

What is claimed is:

1. A computer-implemented method for synchronizing prescription medications for a patient, comprising:
receiving, by a computing system, prescription data associated with a patient;
verifying a use agreement;
authenticating the patient;
determining eligibility for a prescription synchronization program;
receiving an opt-in from the patient; and
synchronizing a plurality of medications according to a computed refill alignment schedule.

2. The method of claim 1, further comprising determining the refill alignment schedule based on at least one of: current refill dates, medication dosages, and refill intervals.

3. The method of claim 1, wherein synchronizing the plurality of medications comprises generating partial fill information for one or more medications to align with other medication refill dates.

4. The method of claim 1, further comprising providing the patient with a confirmation card comprising a pick-up date, pharmacy address, and synchronized medication list.

5. The method of claim 1, wherein authenticating the patient comprises validating inputted identification information against pharmacy records.

6. The method of claim 1, wherein determining eligibility comprises applying patient-level and medication-level exclusion criteria.

7. The method of claim 6, wherein the patient-level exclusion criteria comprise excluding patients who are inactive, deceased, or hospitalized.

8. The method of claim 6, wherein the medication-level exclusion criteria comprise excluding medications with insufficient day supply or lapsed prescriptions.

9. The method of claim 1, further comprising filtering patients who are enrolled, partially enrolled, or have opted out of the synchronization program.

10. The method of claim 1, wherein the use agreement and patient interaction are presented via a chatbot interface communicatively coupled to a patient device.

11. A system for automating prescription medication synchronization, comprising:
a processor;
a memory storing instructions that, when executed by the processor, cause the system to:
retrieve prescription data for a patient;
verify a use agreement;
authenticate the patient;
determine eligibility for a synchronization program;
receive a patient opt-in; and
generate a medication synchronization schedule.

12. The system of claim 11, further comprising a user interface configured to display opt-in options and synchronization details to the patient.

13. The system of claim 11, wherein the synchronization schedule aligns medication refill dates by calculating partial fills.

14. The system of claim 11, wherein the system is configured to exclude patients or medications based on eligibility criteria stored in a pharmacy database.

15. The system of claim 14, wherein the eligibility criteria comprise patient age, enrollment status, activity status, and medication history.

16. The system of claim 11, wherein the system further comprises a chatbot module configured to manage patient interactions and secure identification processes.

17. The system of claim 11, further comprising a medication alignment module configured to compute optimal synchronization dates across multiple prescriptions.

18. The system of claim 11, further comprising a database configured to store confirmation messages including synchronized medication lists and pharmacy pickup instructions.

19. The system of claim 11, wherein the processor is further configured to generate instructions for a pharmacy management system to dispense medications according to the synchronization schedule.

20. The system of claim 11, wherein the system communicates with a cloud-based infrastructure to exchange data with at least one of: a pharmacy system, a patient device, and a healthcare provider system.
